(19) United States
(12) Patent Application Publication
(10) Pub. No.: US 2023/0136060 A1
(43) Pub. Date: May 4, 2023

(54) METHOD FOR PREPARING NANODIAMONDS LABELED WITH RADIOACTIVE GALLIUM

(71) Applicant: Institute of Nuclear Energy Research, Atomic Energy Council, Executive Yuan, R.O.C, Taoyuan (TW)

(72) Inventors: MEI-HUI WANG, Taoyuan (TW); KUN-LIANG LIN, Taoyuan (TW); CHUAN-YI CHIEN, Taoyuan (TW); HUNG-MAN YU, Taoyuan (TW)

(21) Appl. No.: 17/962,614

(22) Filed: Oct. 10, 2022

(30) Foreign Application Priority Data
Nov. 1, 2021 (TW) 110140510

(51) Int. Cl. A61K 51/12; C01B 32/28; A61K 51/04

(52) U.S. Cl. CPC A61K 51/1244; C01B 32/28; A61K 51/0497; C01P 2006/88; C01P 2004/64

(57) ABSTRACT

The method is capable of effectively labeling the nanodiamonds with radioactive gallium and can be operated at room temperature, and therefore is convenient to operate and does not require further purification to obtain the nanodiamonds labelled with radioactive gallium with a purity of at least 99%

### CROSS REFERENCE TO RELATED APPLICATION

This application claims the benefits of Taiwan application Serial No. 110140510, filed on Nov. 1, 2021, the disclosures of which are incorporated by references herein in its entirety.

### BACKGROUND

### Technical Field

The present invention relates to the field of radiolabeling technologies, and in particular, to a method for labeling nanodiamonds with amine surface modification with radioactive gallium.

### Related Art

In this technical field, cell therapy products lack accurate and rapid preclinical cell pharmacokinetic verification. When cells used as drugs (cell drugs) enter an organism, how to effectively perform cell calibration, positioning, quantification, validation, cell dynamics analysis, and the like are all challenges currently faced.

In the related art, fluorescent nanodiamonds are used. After being phagocytosed by cells, the nanodiamonds can effectively stay in the cells, and are not toxic. After the fluorescent nanodiamonds are injected into an organism through cell drugs, the cells contain the nanodiamonds, to facilitate positioning in the organism. Therefore, biodistribution data of the cells in the organism can be obtained, and sequence analysis after cell gene therapy can be further performed in combination with a quantitative polymerase chain reaction (qPCR).

Although fluorescent nanodiamonds may be used to track biological distribution of cells in animals, these animals need to be sacrificed to take out organs to count a quantity of cells, and in vivo imaging observation cannot be performed, leading to restrictions in use of fluorescent nanodiamonds.

In addition, the current method for radiolabeling the nanodiamonds in the related art is restricted in both a procedure and use of radiolabeling, which is not conducive to clinical use. For example, nanodiamonds are labeled with I-125 (Diamond & Related Materials 18 (2009) 95-100). A labeling method of the nanodiamonds requires the use of a strong oxidant chloramine T, and a long period of dialysis is required after the labeling. Finally, the analysis also requires the sacrifice of animals to obtain the distribution of the nanodiamonds. In addition, nanodiamonds are labeled with F-18 (ACS Nano 5(7):5552-5559, 2011). A labeling method of the nanodiamonds first requires preparation and purification of N-Succinimidyl 4-[18F]Fluorobenzoate (¹⁸F-SFB), and then requires centrifugation and separation for labeling of the nanodiamonds. The steps are complex. In addition, there are no related reports of successfully labeling nanodiamonds with a radioactive metal in the related art at present.

In view of this, a novel method for preparing nanodiamonds labeled with radioactive gallium is urgently needed in this technical field, to improve the shortcomings of the related art.

### SUMMARY

In order to make readers understand the basic meaning of the disclosure, the section of Summary provides a brief description of the disclosure. The section of Summary is not a complete description of the disclosure, and it is not intended to define the technical features or the scope of claims of the present invention.

One aspect of the present invention relates to a method for preparing nanodiamonds labeled with radioactive gallium, comprising the following steps:

dissolving a nanodiamond in a triethylamine/dimethylformamide solution, and mixing the solution with thiocyanate-toluene-triazanonane diacetic acid-glutamic acid (p-NCS-Bn-NODA-GA) to obtain a mixture;

ultrasonically vibrating the mixture for at least four hours, removing a supernatant through centrifugation, and performing adding water for redissolution and performing freeze-drying, to obtain a nanodiamond bonded with p-NCS-Bn-NODA-GA; and

adding a sodium acetate solution to the nanodiamond bonded with p-NCS-Bn-NODA-GA, adding a radioactive gallium, and performing reaction at room temperature for 10 minutes to 20 minutes, to obtain a nanodiamond labeled with radioactive gallium without further purification.

In a specific implementation, in step (a) in the method of the present invention, the nanodiamond and the triethylamine/dimethylformamide solution are mixed in a volume ratio of 1:2-4. In another specific implementation, a weight ratio of the nanodiamond and the thiocyanate-toluene-triazanonane diacetic acid-glutamic acid (p-NCS-Bn-NODA-GA) is 0.6-0.8:1. In addition, according to an implementation of the present invention, in step (b), the mixture is ultrasonically vibrated for at least six hours.

In an optional implementation, in step (b), the radioactive metal is gallium-67 or gallium-68. In a preferred implementation, the radioactive metal is Ga-68.

According to another implementation of the present invention, step (c) of the present invention is completed at pH 4-6; and is preferably completed at pH 4-5.

According to still another implementation of the present invention, step (c) is the reaction at room temperature for 15 minutes.

Those of ordinary skill in the art the present invention belongs to can fully learn the central concept, the techniques used and various implementation aspects of the present invention with reference to the detailed description as follows.

### BRIEF DESCRIPTION OF THE DRAWINGS

To make the foregoing and other objectives, features, advantages, and embodiments of the present invention more comprehensible, descriptions of the drawings are as follows:

FIG. 1 is a schematic diagram of a nanodiamond bonded with p-NCS-Bn-DTPA according to an implementation of the present invention.

FIG. 2 is a schematic diagram of a nanodiamond bonded with p-NCS-Bn-NODA-GA according to an implementation of the present invention.

FIG. 3 shows a result of Ga-68 radiolabeled chemical purity test of a nanodiamond bonded with p-NCS-Bn-NODA-GA according to an implementation of the present invention.

### DETAILED DESCRIPTION

To make the description of the disclosure more detailed and complete, the following provides an illustrative text description of the implementation aspects and specific embodiments of the present invention. However, the implementation aspects and specific embodiments of the present invention are not limited thereto.

Unless otherwise stated, the scientific and technical terms used in this specification have the same meanings as those understood and commonly used by a person of ordinary skill in the art. In addition, nouns used in this specification include the singular and plural forms of the nouns, unless otherwise specified.

As described in this specification, the term “about” generally means that an actual value falls within plus or minus 10%, 5%, 1%, or 0.5% of a specific value or range. The term “about” herein means that an actual value falls within an acceptable standard error of an average value, depending on the consideration of a person of ordinary skill in the art the present invention pertains. Except for experiment examples, or unless otherwise clearly stated, it should be understood that the ranges, quantities, numerical values, and percentages used herein are all modified by “about”. Therefore, unless otherwise stated, the values or parameters disclosed in this specification and the appended claims are all approximate values, and may be changed as required.

The term such as “individual” or “patient” refers to an animal capable of being treated with or using nanodiamonds labeled with a radioactive metal of the present invention, including a human. Unless otherwise specified, the term “individual” or “patient” encompasses both male and female animals.

To resolve the problems existing in the related art, the present invention provides a novel method for labeling nanodiamonds with radioactive gallium. It should be noted that, it is difficult to further use p-NCS-Bn-DTPA to modify nanodiamonds with amine surface modification. However, a novel preparation method provided in the present invention can effectively further use p-NCS-Bn-NODA-GA to modify the nanodiamonds with amine surface modification, and radioactive gallium is then radiolabeled. The radiolabeling efficiency is as high as >99%, and the radiolabeling reaction only takes 15 minutes without purification, thereby providing advantages of industrial use.

According to an implementation of the present invention, the method for preparing nanodiamonds labeled with radioactive gallium includes the following steps:

(a) dissolving a nanodiamond in a triethylamine/dimethylformamide solution, and mixing the solution with thiocyanate-toluene-triazanonane diacetic acid-glutamic acid (p-NCS-Bn-NODA-GA) to obtain a mixture;

(b) ultrasonically vibrating the mixture for at least four hours, removing a supernatant through centrifugation, and performing adding water for redissolution and performing freeze-drying, to obtain a nanodiamond bonded with p-NCS-Bn-NODA-GA; and

(c) adding a sodium acetate solution to the nanodiamond bonded with p-NCS-Bn-NODA-GA, adding a radioactive gallium, and performing reaction at room temperature for 10 minutes to 20 minutes, to obtain a nanodiamond labeled with radioactive gallium without further purification.

According to an implementation, in step (a), the nanodiamond and the triethylamine/dimethylformamide solution were mixed in a volume ratio of about 1:2-4. For example, the volume ratio is about 1:2, 1:3, and 1:4.

In another implementation, a weight ratio of the nanodiamond and the thiocyanate-toluene-triazanonane diacetic acid-glutamic acid (p-NCS-Bn-NODA-GA) is about 0.6-0.8:1, for example, about 0.6:1, 0.7:1, and 0.8:1.

According to an implementation, the mixture was ultrasonically vibrated for at least six hours, for example, about 6, 7, 8, 9, or 10. In a preferred implementation, the mixture was ultrasonically vibrated for six hours.

In addition, the radioactive gallium added in the present invention is gallium-67 or gallium-68. Those with general knowledge in the technical field may select a suitable radioactive metal for labeling according to actual use requirements. Moreover, in a preferred implementation, the radioactive metal is Ga-68.

According to an implementation of the present invention, in step (c) of the present invention, a sodium acetate solution was added so that the reaction was carried out at a pH of about 4-6, preferably a pH of 4-5, and more preferably a pH of about 4.5.

In an optional implementation, step (c) was the reaction at room temperature for 10 to 20 minutes, for example, about 10, 11, 12, 13, 14, 15, 16, 17, 18, 19, or 20 minutes, preferably about 15 minutes.

A plurality of embodiments is disclosed below to illustrate various different implementation aspects of the present invention, so that those with general knowledge in the technical field of the present invention can implement the technical content disclosed in the present invention according to the disclosure of the specification. Therefore, the embodiments disclosed below cannot be used to limit the scope of rights of the present invention. Furthermore, all documents cited in the specification are deemed to be fully cited as a part of the specification.

### Embodiment 1: Bonding Between Nanodiamonds and p-NCS-Bn-DTPA

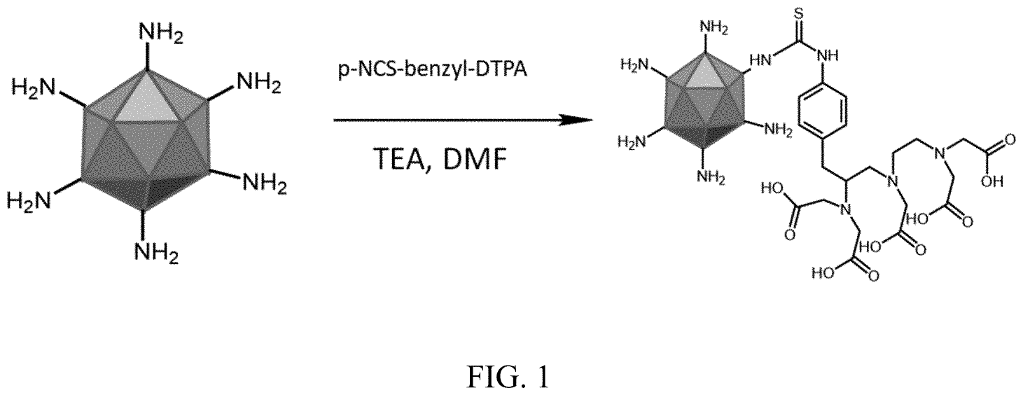

Nanodiamonds (5 mg) were dissolved in triethylamine/dimethylformamide (0.3 mL/3 mL), thiocyanate-toluene-diethylenetriaminepentaacetic acid (p-NCS-benzyl-DTPA, 5 mg Macrocyclics, USA. FW=649.9) was added, and ultrasonically vibrating and stirring were performed for six hours. Centrifugation was performed at 14,000 rpm for five minutes, and a supernatant was removed. The precipitate was insoluble in 0.5 mL of deionized water and insoluble in dimethylformamide, methanol, and dimethyl sulfoxide, and cannot be measured by using quantitative nuclear magnetic resonance (qNMR) for measurement. Therefore, the following In-111 radiolabeling was directly performed. A nanodiamond bonded with p-NCS-Bn-DTPA is shown in FIG. 1.

### Embodiment 2: In-111 Radiolabeling of a Nanodiamond Bonded with p-NCS-Bn-DTPA

20 μL of acetonitrile, methanol, ethanol, dimethylformamide, and acetone were respectively added to the nanodiamond bonded with p-NCS-Bn-DTPA obtained in Embodiment 1; 1 μL was taken, 10 μL of sodium citrate (pH 4) was added, and 1 μL of In-111 (activity of about 24 uCi) was added, reaction was performed at room temperature for 15 minutes; and radioactive thin-layer chromatography was performed with 0.1 M EDTA. The In-111 radiolabeled chemical purities thereof are respectively 24%, 15%, 30%, 19%, and 12%. For an In-111 radiolabeled chemical purity test method for the nanodiamond bonded with p-NCS-Bn-DTPA, reference is made to Embodiment 3.

### Embodiment 3: In-111 Radiolabeled Chemical Purity Test Method for the Nanodiamond Bonded with p-NCS-Bn-DTPA

10 mL of 0.1 M EDTA solution was added to a developing tank, and an origin and a solvent front were respectively marked at 1 cm and 5 cm of an RP-TLC film. The sample (1 μL) was dropped at the origin of the RP-TLC film, and was placed in the developing tank with a tweezer. When the developing solution reaches the solvent front, the sample was taken out with the tweezer and dried. The RP-TLC film was scanned with a radio-TLC image scanner, and a spectrum was collected for one minute. A result calculation method is as follows:

A: The count area of the In-111-nanodiamond-NCS-Bn-DTPA peak (Rf=0.0-0.2)

B: The count area of all peaks

### Embodiment 4: Bonding Between Nanodiamonds and p-NCS-Bn-NODA-GA

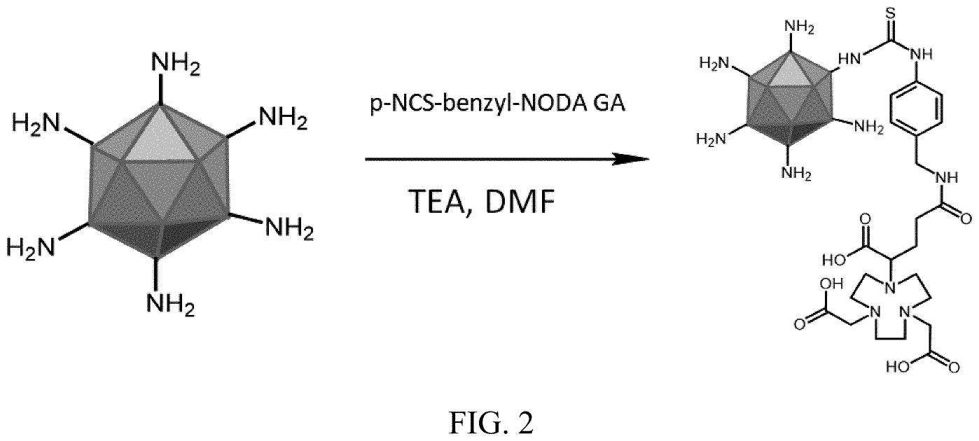

Nanodiamonds (1.4 mg) were dissolved in triethylamine/dimethylformamide (0.3 mL/3 mL), thiocyanate-toluene-triazanonane diacetic acid-glutamic acid (p-NCS-benzyl-NODA GA, 1 mg CheMatech, France. FW=521.59) was added, and ultrasonically vibrating and stirring were performed for six hours. Centrifugation was performed at 14,000 rpm for five minutes, and a supernatant was removed. After 0.5 mL of deionized water was added, the precipitate can no longer be obtained by centrifugation at 14,000 rpm for five minutes, and can only be freeze-dried. Because a product of the bonding between the nanodiamonds and p-NCS-Bn-NODA-GA was water-soluble, a signal on a benzene ring of p-NCS-Bn-NODA-GA may be obtained through qNMR by using butenedioic acid as an internal standard product. After calculation compared with the internal standard product, it may be learned that 0.8 mg of nanodiamonds bonded with p-NCS-Bn-NODA-GA was obtained, as shown in FIG. 2.

### Embodiment 5: Ga-68 Radiolabeling of a Nanodiamond Bonded with p-NCS-Bn-NODA-GA

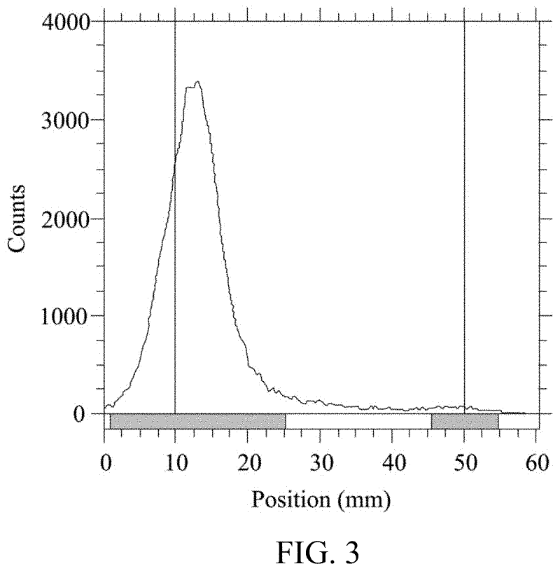

0.8 mg of nanodiamonds bonded with p-NCS-Bn-NODA-GA was lyophilized and dissolved in 20 μL of deionized water, 4 μL was added to 166 μL of 1 M of sodium acetate, a pH value of the mixture was adjusted to about 4.5, and 0.5 mL of Ga-68 (activity about 5 mCi) (final pH=4.5) was then added, reaction was performed at room temperature for 15 minutes to obtain nanodiamonds labeled with a radioactive metal of the present invention (Ga-68 of the nanodiamonds bonded with p-NCS-Bn-NODA-GA); and radioactive thin-layer chromatography was performed with 0.1 M EDTA. The Ga-68 radiolabeled chemical purity thereof is 99.46% (as shown in FIG. 3). For a Ga-68 radiolabeled chemical purity test method for the nanodiamond bonded with p-NCS-Bn-NODA-GA, reference is made to Embodiment 6.

### Embodiment 6: Ga-68 Radiolabeled Chemical Purity Test Method for the Nanodiamond Bonded with p-NCS-Bn-NODA-GA

10 mL of 0.1 M EDTA solution was added to a developing tank, and an origin and a solvent front were respectively marked at 1 cm and 5 cm of an RP-TLC film. A small amount of the sample (1-2 μL) was dropped at the origin of the RP-TLC film, and was placed in the developing tank with a tweezer. When the developing solution reaches the solvent front, the sample was taken out with the tweezer and dried. The RP-TLC film was scanned with a radio-TLC image scanner, and a spectrum was collected for one minute. A result calculation method is as follows:

A: The count area of the Ga-68-nanodiamond-NCS-Bn-NODA-GA peak (Rf=0.0-0.2)

B: The count area of all peaks

The results of Embodiments 1 to 3 above show that, it is difficult to further use p-NCS-Bn-DTPA to modify nanodiamonds with amine surface modification, and even with In-111 radiolabeling, high radiolabeling efficiency cannot be achieved. In contrast, the method of the present invention, that is, the results of Embodiments 4 to 6, can confirm that the method of the present invention can be used for labeling of radioactive nucleus gallium, and the labeling can be completed through reaction for 15 minutes at room temperature with no further purification. The labeling technology of metal radioactive nucleus gallium of nanodiamonds of the present invention has advantages, and the radiolabeling efficiency is as high as >99%. Gallium-68 (with a half-life of 68 minutes) and Gallium-67 (with a half-life of 67.2 hours) are isotopes of each other and have the same chemical properties. After nanodiamonds are radiolabeled with Gallium-68 and Gallium-67 and phagocytosed, changes in in vivo imaging for a short time and for a long time may be observed separately. Further, due to the short half-life of Ga-68 (68 minutes), there is no radioactivity in the living body after the imaging. However, the long half-life of Ga-67 (78.2 hours) has the advantage of long time observation of distribution of cell organisms. In short, the method for preparing nanodiamonds labeled with radioactive gallium has advantages of industrial use as a cell tracer in future clinical cell therapy.

What is claimed is:

1. A method for preparing nanodiamonds labeled with radioactive gallium, comprising:
(a) dissolving a nanodiamond in a triethylamine/dimethylformamide solution, and mixing the solution with thiocyanate-toluene-triazanonane diacetic acid-glutamic acid (p-NCS-benzyl-NODA GA) to obtain a mixture;
(b) ultrasonically vibrating the mixture for at least four hours, removing a supernatant through centrifugation, and performing adding water for redissolution and performing freeze-drying, to obtain a nanodiamond bonded with p-NCS-Bn-NODA-GA; and
(c) adding a sodium acetate solution to the nanodiamond bonded with p-NCS-Bn-NODA-GA, adding a radioactive gallium, and performing reaction at room temperature for 10 minutes to 20 minutes, to obtain a nanodiamond labeled with radioactive gallium without further purification.

2. The method according to claim 1, wherein in step (a), the nanodiamond and the triethylamine/dimethylformamide solution are mixed in a volume ratio of 1:2-4.

3. The method according to claim 1, wherein in step (a), a weight ratio of the nanodiamond and the thiocyanate-toluene-triazanonane diacetic acid-glutamic acid (p-NCS-benzyl-NODA GA) is 0.6-0.8:1.

4. The method according to claim 1, wherein in step (b), the mixture is ultrasonically vibrated for at least six hours.

5. The method according to claim 1, wherein in step (b), the radioactive metal is Ga-67.

6. The method according to claim 1, wherein in step (b), the radioactive metal is Ga-68.

7. The method according to claim 1, wherein step (c) is completed at pH 4-6.

8. The method according to claim 7, wherein step (c) is completed at pH 4-5.

9. The method according to claim 1, wherein step (c) is the reaction at room temperature for 15 minutes.

10. The method according to claim 1, wherein the nanodiamond bonded with p-NCS-Bn-NODA-GA is a water-soluble nanoparticle.

11. The method for preparing nanodiamonds labeled with radioactive gallium according to claim 1, wherein the method does not require any purification steps.
